(19) United States
(12) Patent Application Publication
(10) Pub. No.: US 2014/0277114 A1
(43) Pub. Date: Sep. 18, 2014

(54) VASCULAR CLOSURE DEVICE WITH OCCLUSION BALLOON GUIDEWIRE

(71) Applicant: Essential Medical, Inc., Malvern, PA (US)

(72) Inventor: Greg Walters, Exton, PA (US)

(73) Assignee: Essential Medical, Inc., Malvern, PA (US)

(21) Appl. No.: 14/199,271

(22) Filed: Mar. 6, 2014

Related U.S. Application Data
(60) Provisional application No. 61/794,069, filed on Mar. 15, 2013.

(51) Int. Cl. A61B 17/00

(52) U.S. Cl. CPC A61B 17/0057
USPC 606/213

(57) ABSTRACT

A method of sealing a vascular puncture of a vessel can include the step of inserting a guidewire through a sheath and into the vessel. The guidewire can include a hollow tube and a balloon coupled to a distal end of the hollow tube. The method can further include the steps of inflating the balloon so as to temporarily occlude the vessel and guiding a closure device along the hollow tube toward the puncture such that a toggle of the closure device enters the vessel through the puncture; and sealing the puncture with the closure device.

### CROSS REFERENCE TO RELATED APPLICATIONS

This application claims priority to U.S. Provisional Application Ser. No. 61/794,069, filed Mar. 15, 2013, the contents of which are hereby incorporated by reference as if set forth in their entirety herein.

### BACKGROUND

During large bore procedures, sheath exchange or removal can lead to a significant loss of blood. One way this may be mitigated is to utilize temporary balloon occlusion of the common femoral artery proximal to the sheath site, typically accessed from a contralateral femoral site. A simple low pressure inflation of the balloon creates flow stasis, allowing for bloodless sheath removal or exchange. However, a contralateral balloon placement is not always possible in the setting of iliac disease, as often covered stents descend into the iliac vessels and thus prevent access to the iliac bifurcation and the opposite leg artery. It may therefore be desirable to make it possible to manage flow while at the same time providing for an iliac prosthesis.

### SUMMARY

In accordance with an embodiment, a vascular closure system configured to seal a puncture in a vessel can include a guidewire and a closure device. The guidewire can be configured to be inserted through the puncture and into the vessel, and can have a hollow tube and an inflatable balloon coupled to a distal end of the hollow tube. The inflatable balloon can be configured to occlude the vessel. The closure device can include a toggle, a plug, and a filament that couples the toggle to the plug. The toggle and plug each can define a respective guidewire channel that is configured to receive the hollow tube such that the closure device is advanceable along the hollow tube toward the puncture.

In one embodiment, a method of sealing a vascular puncture of a vessel can include the steps of inserting a guidewire through a sheath and into the vessel, the guidewire including a hollow tube and a balloon coupled to a distal end of the hollow tube; inflating the balloon so as to temporarily occlude the vessel; guiding a closure device along the hollow tube toward the puncture such that a toggle of the closure device enters the vessel through the puncture; and sealing the puncture with the closure device.

In another embodiment, a method of sealing a vascular puncture of a vessel with a closure device having a toggle and a plug, can include the steps of inserting a guidewire through a sheath and into the vessel, the guidewire including a hollow tube and a balloon coupled to a distal end of the hollow tube; inflating the balloon so as to temporarily occlude the vessel; advancing the hollow tube through the toggle and through the plug; guiding the closure device along the hollow tube toward the puncture; and sealing the puncture with the toggle and the plug.

### BRIEF DESCRIPTION OF THE DRAWINGS

The foregoing summary, as well as the following detailed description of an example embodiment of the application, will be better understood when read in conjunction with the appended drawings, in which there is shown in the drawings example embodiments for the purposes of illustration. It should be understood, however, that the application is not limited to the precise arrangements and systems shown. In the drawings:

FIG. 1 is a perspective view of a puncture sealing device in accordance with an embodiment, the puncture sealing device being slidable along a guide wire and having a deployment device and a closure device disposed within the deployment device;

FIG. 2 is a sectional view showing a toggle of the closure device trapped between a release tube of the deployment device and a delivery tube of the deployment device;

FIG. 3A is a schematic showing a puncture locating dilator positioned such that the inlet hole is disposed within a vessel proximate to a puncture site and such that an external radiopaque marker placed on the skin is to be aligned with the radiopaque marker on the dilator body;

FIG. 3B is a schematic showing a procedure sheath inserted into the vessel and the puncture locating dilator removed;

FIG. 3C is a schematic showing the guidewire of FIG. 1 having a distal balloon and being inserted through an access channel of the procedure sheath;

FIG. 3D is a schematic showing the balloon of FIG. 3C being inflated with an inflation device;

FIG. 3E is a schematic showing the inflated balloon of FIG. 3D after the inflation device has been removed;

FIG. 3F is a schematic showing the closure device translated along the guidewire of FIG. 3E and into an access channel of the access sheath such that a distal end of the toggle of the closure device is positioned distal to a distal end of the access sheath;

FIG. 3G is a schematic showing the access sheath and closure device combination pulled proximally along the guide wire such that the toggle is proximate to the puncture site;

FIG. 3H is a schematic showing the release tube being moved proximally relative to the delivery tube to thereby release the toggle;

FIG. 3I is a schematic showing a plug of the closure device being pressed against the vessel wall with a locking member while the guidewire remains in place; and

FIG. 3J is a schematic showing the puncture site fully sealed with the guidewire removed.

### DETAILED DESCRIPTION OF ILLUSTRATIVE EMBODIMENTS

Certain terminology is used in the following description for convenience only and is not limiting. The words “right”, “left”, “lower” and “upper” designate directions in the drawings to which reference is made. The words “proximally” and “distally” refer to directions toward and away from, respectively, the individual operating the system. The terminology includes the above-listed words, derivatives thereof and words of similar import.

Referring to FIGS. 1 and 2 a puncture sealing device 10 in accordance with an embodiment of the invention can include a deployment device 14 and a closure device 18 at least partially disposed within the deployment device 14. After the deployment device is inserted into a vessel through a puncture site of the vessel, the closure device 18 is deployed from the deployment device 14 to thereby seal or otherwise close the puncture site of the vessel. As shown in FIG. 3F, the puncture sealing device can be guided along a guidewire 72 that extends from within the vessel through the puncture site and out of the patient's body. As shown in FIG. 3F, the guidewire 72 can include a hollow tube 500, an inflatable balloon 510 coupled to a distal end of the hollow tube 500, a floppy tip 520 that extends distally from the inflatable balloon, and a guidewire valve 530 disposed at a proximal end of the hollow tube 500. The balloon 510 is configured to be inflated prior to the sealing device being moved along the hollow tube 500 toward the vessel. The balloon 510 may be manufactured from any suitable elastomeric rubber-like compound such as natural rubber latex, isoprene, neoprene, butyl rubber, or silicone.

As shown in FIG. 2 the deployment device 14 includes a release tube 22 that is elongate along a first direction L and defines a release tube channel 26 that extends through the release tube 22 along the first direction L. The release tube 22 is configured to restrain a toggle 40 of the closure device 18 during insertion of the sealing device into the vessel and subsequently release the toggle 40 so that the toggle 40 can be oriented for the sealing procedure. As shown in FIG. 2, the deployment device 14 further includes a delivery tube 30 that is disposed within the release tube channel 26 such that at least one of the release tube 22 and the delivery tube 30 is movable relative to the other along the first direction L. Therefore, the release tube 22 and the delivery tube 30 can be configured such that at least one of the release tube 22 and the delivery tube 30 is movable relative to the other to thereby release the toggle 40 and subsequently orient the toggle 40 for the sealing procedure.

As shown in FIG. 2, the delivery tube 30 includes an angled portion 31 at its distal end. The angled portion 31 angles toward a central axis of the delivery tube 30 such that a retention cavity 32 is defined between the angled portion 31 and the release tube 22. The retention cavity 32 is sized to receive and retain a portion of the toggle 40 to thereby trap the toggle 40 between the delivery tube 30 and the release tube 22 such that the toggle 40 is angled by a first angle Ø₁ relative to a central axis of the release tube 22. While the toggle 40 is trapped, the closure device 18 and deployment device 14 can be inserted into the vessel.

As shown in FIG. 2, the closure device 18 is at least partially disposed within the delivery tube 30 prior to being inserted into the vessel. As shown in FIGS. 1 and 2, the closure device 18 further includes a plug 44 (e.g. collagen pad), a locking member 48, and a filament 52 that couples the toggle 40, plug 44, and locking member 48 together such that the toggle 40 is distal to the plug 44 and the locking member 48 is proximal to the plug 44. As shown in FIG. 1, the filament 52 extends through the locking member 48, plug 44, and toggle 40 in the first direction L and then back through the toggle 40 and plug 44 in a direction opposite the first direction L. An end of the filament 52 is then formed into a slidable knot 56 that is slidable along the filament 52 between the plug 44 and the locking member 48. In operation, the locking member 48 and toggle 40 squeeze the plug 44 against the puncture site to thereby seal the puncture site.

The toggle 40 can be an elongate, low profile member that is configured to be seated inside the vessel against the vessel wall contiguous with the puncture site. The toggle 40 defines a distal end 40a that is distal to a distal end of the release tube 22 and a proximal end 40b that is trapped within the retention cavity 32 between the release tube 22 and the delivery tube 30 during insertion of the toggle 40 into the vessel. As shown in FIG. 1, the toggle further defines a first filament receiving aperture 60 that receives the filament 52 as it passes through the toggle 40 in the first direction L, a second filament receiving aperture 64 that receives the filament 52 as it passes through the toggle 40 in the second direction, and a guidewire aperture 68 that is configured to receive the guidewire 72 such that the closure device 18 translates along the hollow tube 500 of the guidewire 72 and is guided toward the puncture site by the guidewire 72. The toggle 40 can be made of any desired material. For example, the toggle 40 can made of a polylactic-coglycolic acid or other synthetic absorbable polymer that degrades in the presence of water into naturally occurring metabolites. It should be appreciated, however, that the toggle 40 can be made of other materials and can have other configurations so long as it can be seated inside the vessel against the vessel wall.

With continued reference to FIG. 1, the plug 44 is coupled to the filament 52 between the toggle 40 and the locking member 48. Like the toggle 40, the plug 44 can have a series of filament receiving apertures 76 that receive the filament 52 along the first and second directions to thereby couple the plug 44 to the filament. The plug 44 can further include a series of guidewire apertures 80 that receive the hollow tube 500 of the guidewire 72 during insertion of the closure device 18 into the vessel. The plug 44 can comprise a strip of compressible, resorbable, collagen foam and can be made of a fibrous collagen mix of insoluble and soluble collagen that is cross linked for strength. It should be appreciated, however, that the plug 44 can have any configuration as desired and can be made from any material as desired.

With continued reference to FIG. 1, the locking member 48 is configured to frictionally engage the filament 52 as the locking member 48 is moved along the filament 52 toward the toggle 40 to thereby seal the puncture site. That is, the locking member 48 is configured to remain in place on the filament 52 when no force is placed on the locking member 48, and only overcomes its frictional engagement with the filament 52 in response to an application of force on the locking member 48. The locking member 48 can be configured as a cylindrical member that is crimped onto the filament 52. It should be appreciated, however, that the locking member 48 can have other configurations as desired. For example, the locking member 48 can be the slideable knot 56. In such an embodiment, the slidable knot 56 can be a locking knot.

As shown in FIG. 1, the closure device 18 further includes a tamper 90 proximal to the locking member 48 and a tensioning device 94 proximal to the tamper 90. As shown, the guidewire 72 and the filament 52 extend through both the tamper 90 and the tensioning device 94. The tamper 90 is configured to be translated along the filament 52 to thereby move the locking member 48 against the plug 44. In this way, the puncture site can be fully sealed. The tensioning device 94 is configured to maintain the filament 52 in tension during the sealing procedure.

Embodiments of the present technology will now be described with respect to exemplary large bore procedures that utilize the puncture sealing device 10. In order to perform any of the related procedures, the user gains percutaneous access to, for example, the femoral artery, causing a puncture site in the artery. To gain percutaneous access to the artery, the Seldinger technique may be used. For example, a hollow bore needle is inserted into the artery. A guidewire 200 is then advanced through the hollow needle and into the femoral artery a sufficient distance to allow removal of the needle without the guidewire 200 pulling out of the vessel. Removing the needle leaves the guidewire in place, with a portion of the guidewire extending into the artery. The guidewire 200, extending from outside the patient into the femoral artery, provides for an entry guide for other medical devices. Therefore, once the guidewire 200 is positioned in the vessel of the patient, catheters, or introducers, or gradually increasing diameters are advanced over the guidewire 200 and through the puncture into the artery to further open the puncture site.

Now referring to FIG. 3A, a proximal end of the guidewire 200 can be inserted into the distal end of the puncture locating dilator 310. As shown in FIG. 3A, the puncture locating dilator 310 can then be moved along the guidewire 200 until the distal end of the puncture locating dilator 310 and the blood inlet hole 320 enter the vessel 120 such that blood flows into the inlet hole 320 and out the outlet hole 340 to thereby locate a position of the puncture site 130. The position of the puncture site 130 can be confirmed via feedback of blood flow exiting the blood outlet hole 340 by alternatingly inserting and retracting the puncture locating dilator 310. As shown in FIG. 3A, after the position of the puncture site 130 has been located, a position of the radiopaque marker 330 of the dilator 310 can be determined on an imaging device and an external marker 140 can be positioned on the patient. The external marker 140 can be positioned such that it corresponds with the position of the radiopaque marker 330 of the dilator 310 to thereby provide a visual indication of the puncture site location after the dilator 310 is removed from the guidewire 200. It should be appreciated, that in some embodiments, the puncture locating dilator 310 can be positioned over the guidewire prior to the guidewire being inserted into the vessel 120.

Referring to FIG. 3B, after all dilating catheters are utilized and removed from the guidewire 200, the guidewire 200 will remain in the vessel and protrude out of the patient and be accessible externally as an access guide (often termed as a rail, wire guide or the like). At this stage, an introducer set that can include a large bore introducer and access sheath 400, ranging in size from, for example, 14-22F and intended for using during the large bore intervention, is advanced over the guidewire 200 and into the vessel until the access sheath 400 is fully inserted to the users' satisfaction. Once the user is satisfied regarding position, the introducer and guidewire 200 are removed, leaving only the access sheath 400 within the vessel as is depicted in FIG. 3B.

As shown, the access sheath 400 can include a sheath shaft 430, a hub 410 generally containing a hemostasis valve (not shown), and a sideport with stopcock 420. The sheath provides access for catheters, for example, 14-22F, and thereby has internal dimensions of shaft 430 and hub 410 sized accordingly. Thus, the outer diameter of the shaft 430 can be larger than the indicated size, and as is the case with some procedure introducer sets commonly available, may be considerably larger that the inner diameter (i.e. the wall thickness of the access sheath shaft 430 can be considerable). It should be appreciated, that the external radiographic marker 140 remains in position and is unobtrusive to the user. After positioning of the access sheath 400, the larger bore intervention is then conducted. For example, procedures may include aortic balloon valvuloplasty (BAV) for the treatment of aortic valve disease, endovascular prothesis placement (EVAR) for the treatment of abdominal aorta disease or the like (abdominal aortic aneurysm repair, or AAA), and/or the trans-catheter placement of stent valves for the replacement of damaged or diseased aortic valves (trans-catheter aortic valve implantation, TAVI). Once an interventional procedure of this nature is completed, the treatment catheters and other hardware utilized is removed from the access sheath 400, and the access sheath 400 remains as shown in FIG. 3B until it is desired to remove the access sheath 400 and close the puncture 130, which is oftentimes done through surgical means via a cutdown through the skin 100 and subcutaneous tissue 110. To further advance the state of the art, a method for large bore puncture closure that minimizes blood loss, or the complexity of the above procedures is desirable.

Such a method involves the use of a balloon protection guidewire system as shown in its first steps of use in FIGS. 3C-3E. As shown in FIG. 3C, the hollow balloon protection guidewire 72 is inserted through the access sheath 400 and into the vessel 120 with the balloon 510 deflated. Once inserted, an inflation system 600 is attached to the guidewire valve 530. In particular, the inflation system 600 can include a syringe 610 filled with gas or saline (not shown), a plunger 620, and a connection valve 630 (typically referred to as a touhy borst valve), and the inflation system 600 is attached to the guidewire valve 530 via the touhy borst valve 630. As shown in FIG. 3D, the guidewire balloon 510 is inflated by depressing the syringe plunger 620. The balloon and syringe contents are sized such that when the balloon is inflated it will fully occlude, but not over pressurize the vessel, in a manner commonly referred to as ‘low pressure inflation.’

At this stage blood flowing in the vessel will have stopped, and in the case of use of the system whereby the sheath and guidewire are oriented in a retrograde (or pointed against flow) fashion in the femoral artery, the balloon 510 will have been inflated proximally to the puncture site and thus will prevent blood flow to the sheath and puncture. This balloon occlusion can be checked either by injection of contrast media through the sheath sideport 420 and fluoroscopic exam, or by simply opening the sheath sideport and observing the flow of blood from the sideport. No flow out of the sideport indicates occlusion, flow indicates incomplete occlusion. In the case of injecting contrast media and checking fluoroscopically, the contrast injection will fill the vessel to the edge of the inflated balloon and ‘overflow’ into the distal portion of the vessel. This contrast bolus will be washed away by flowing blood, therefore, unless the balloon is fully occlusive, a static column of contrast indicates balloon occlusion. Once the balloon is inflated and occlusion is confirmed, the guidewire valve 530 may be set, which allows removal of inflation system 600 while still maintaining balloon inflation. The valve may work according to any method that utilizes a low-profile valve of outside diameter substantially similar to the outside diameter of the guidewire. Once the valve is set, the procedure access sheath 400 is removed from the guidewire leaving the inflated balloon and guidewire within the vessel, with the hollow tube of the guidewire 500 extending to the outside of the vessel and externally past the skin 100 of the patient.

The user now advances the closure device introducer set (not shown) over the guidewire and into the puncture. Once the closure device sheath set is positioned, the closure introducer (also not shown) is removed from the closure device access sheath 1400 and removed from the guidewire. Thus, as is shown in FIG. 3E, the procedure access sheath 400 was ‘exchanged’ for the closure device sheath 1400, which has elements common to a sheath such as the shaft 1430, and the hub 1410. Note that the guidewire 72 is still in place with the occlusion balloon 510 still inflated as to provide vessel occlusion, and the external radiographic marker 140 is in place as before. Referring to FIG. 3F, closure device sheath hub 1410 is specifically designed to interface with the closure device delivery system 10. As shown in FIG. 3F, the closure device is guided along the hollow tube 500 toward the puncture through the closure device sheath 1400 until the closure device is attached (snapped) to the closure device sheath hub 1410 as indicated by the arrow in the figure. Also shown protruding distally out of the closure device sheath shaft 1410 is the distal portion of the closure device system, including the toggle 40 and release tube 22. Note that at least the toggle 40 is over the wire 72, preferably at least the toggle 40 and plug 44 are over the wire 72, and even more preferable, at least the toggle 40, the plug 44, and the tamper 90 are over the wire 72 during insertion of the closure device.

Once the closure device delivery system 10 has been inserted into the sheath 1400 and snapped to the sheath hub 1410, the entire combined assembly is withdrawn from the patient under fluoroscopic guidance, as shown in FIG. 3G according with the large arrow, until the release tube radiographic marker 631 aligns the external radiographic marker 140, i.e. the markers overlap or align on the fluoroscope image. This action places the toggle 40, as shown, in near proximity of the puncture 130, and further, given the manner in which the guidewire 72 and sheath shaft 1430 are forced to curve into the puncture, biases the distal portion of the closure delivery system, i.e. the release tube 22, toggle 40, or guidewire 72 is or are forced against the posterior vessel wall. Further, the release tube 22 is physically connected to a release knob 650 of the deployment device 14, whereby a pulling motion on the release knob 650 relative to the toggle 40 and delivery tube 30 as depicted by the large arrow in FIG. 3H, moves the release tube 22 backward (upward, or proximally) relative to the toggle 40 and delivery tube 30, and thereby releases the toggle 40 into an orientation largely parallel with the vessel 120 owing to the force bias of the curved components. These actions are indicated by the small arrows. Given the proximity of the toggle 40 relative to the puncture 130, further withdraw of the combined assembly serves to move the toggle 40 into position on the inside surface of the vessel 120 at the puncture site 130 as intended. The final position of toggle 40 is depicted in FIG. 3H after complete deployment of the closure components.

Referring further to FIG. 3I, the closure device 10 is shown deployed such that the closure device 10 has sealed the puncture 130. In particular, the toggle 40 is abutting an inner surface of the vessel wall 120, the plug 44 is compressed against an outer surface of the vessel wall 120, and the lock 48 is holding the compressed plug 44 against the outer surface of the vessel wall 120. The occlusion balloon 510 can remain inflated until the user has completely deployed the components as shown in the FIG. 3I, at which time the user deflates the balloon, and as is depicted by the arrow, removes the guidewire 72 including the balloon 510 from the closure device 10. In particular the guidewire 72 including the balloon 510 is pulled proximally through the toggle 40 and through the plug 44. The user may adjust the plug 44 around the exit hole of the guidewire balloon 510 as may be necessary, after which the puncture 130 is sealed and the filament 52 is cut to below the skin level as pictured as shown in FIG. 3J.

While the foregoing description and drawings represent the preferred embodiment of the present invention, it will be understood that various additions, modifications, combinations and/or substitutions may be made therein without departing from the spirit and scope of the invention as defined in the accompanying claims. In particular, it will be clear to those skilled in the art that the invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components, which are particularly adapted to specific environments and operative requirements without departing from the principles of the invention. In addition, features described herein may be used singularly or in combination with other features. For example, features described in connection with one component may be used and/or interchanged with features described in another component. The presently disclosed embodiment is therefore to be considered in all respects as illustrative and not restrictive, the scope of the invention being indicated by the appended claims, and not limited to the foregoing description.

It will be appreciated by those skilled in the art that various modifications and alterations of the invention can be made without departing from the broad scope of the appended claims. Some of these have been discussed above and others will be apparent to those skilled in the art.

What is claimed:

1. A method of sealing a vascular puncture of a vessel, the method comprising the steps of:
inserting a guidewire through a sheath and into the vessel, the guidewire including a hollow tube and a balloon coupled to a distal end of the hollow tube;
inflating the balloon so as to temporarily occlude the vessel;
guiding a closure device along the hollow tube toward the puncture such that a toggle of the closure device enters the vessel through the puncture; and
sealing the puncture with the closure device.

2. The method of claim 1, further comprising the step of deflating the balloon after the sealing step.

3. The method of claim 2, further comprising the step of pulling the guidewire proximally such that the guidewire is removed from the closure device.

4. The method of claim 1, wherein the inflating step comprises the steps of:
attaching an inflation system to the hollow tube; and
injecting a gas or saline into the balloon with the inflation system.

5. The method of claim 4, wherein the injecting step comprises the step of:
depressing a syringe plunger of the inflation system.

6. The method of claim 4, further comprising the step of:
determining whether the vessel is occluded by opening a valve of the sheath.

7. The method of claim 4, further comprising the step of:
removing the inflation system prior to the guiding step.

8. The method of claim 7, further comprising the step of:
closing a valve on the guidewire prior to the removing step.

9. The method of claim 1, wherein the closure device further includes a plug, a lock, and a filament that couples the toggle, plug, and lock to each other, and wherein the sealing step comprises:
abutting the toggle against an inner surface of the vessel;
compressing the plug against an outer surface of the vessel; and
holding the compressed plug against the outer surface with the lock.

10. The method of claim 9, further comprising the step of:
pulling the guidewire proximally through the toggle and through the plug after the sealing step.

11. The method of claim 1, wherein the closure device is slid onto the hollow tube after the inserting step.

12. A method of sealing a vascular puncture of a vessel with a closure device having a toggle and a plug, the method comprising the steps of:
inserting a guidewire through a sheath and into the vessel, the guidewire including a hollow tube and a balloon coupled to a distal end of the hollow tube;
inflating the balloon so as to temporarily occlude the vessel;
advancing the hollow tube through the toggle and through the plug;
guiding the closure device along the hollow tube toward the puncture; and
sealing the puncture with the toggle and the plug.

13. The method of claim 12, further comprising the steps of:
deflating the balloon after the sealing step; and
pulling the guidewire proximally such that the guidewire is removed from the toggle and the plug.

14. The method of claim 13, wherein the inflating step comprises the steps of:
attaching an inflation system to the hollow tube; and
injecting a gas or saline into the balloon with the inflation system.

15. The method of claim 14, wherein the injecting step comprises the step of:
depressing a syringe plunger of the inflation system.

16. The method of claim 15, further comprising the step of:
removing the inflation system prior to the advancing step.

17. The method of claim 13, wherein the closure device further includes a lock and a filament that couples the toggle, plug, and lock to each other, and wherein the sealing step comprises:
abutting the toggle against an inner surface of the vessel;
compressing the plug against an outer surface of the vessel; and
holding the compressed plug against the outer surface with the lock.

18. A vascular closure system configured to seal a puncture in a vessel, the system comprising:
a guidewire configured to be inserted through the puncture and into the vessel, the guidewire having a hollow tube and an inflatable balloon coupled to a distal end of the hollow tube, the inflatable balloon being configured to occlude the vessel;
a closure device including a toggle, a plug, and a filament that couples the toggle to the plug, the toggle and plug each defining a respective guidewire channel that is configured to receive the hollow tube such that the closure device is advanceable along the hollow tube toward the puncture.

19. The vascular closure system of claim 18, wherein the guidewire channels of the plug and toggle are sized such that the inflatable balloon is movable through the guidewire channels when the guidewire is pulled proximally relative to the closure device after the closure device has sealed the puncture and the inflatable balloon has been deflated.

20. The vascular closure system of claim 18, wherein the guidewire further includes a floppy tip that extends distally from the inflatable balloon.

21. The vascular closure system of claim 18, wherein the guidewire includes an inflation system configured to be coupled to a proximal end of the hollow tube, the inflation system being configured to inject a gas or saline into the inflatable balloon.
